(19) United States
(12) Patent Application Publication
(10) Pub. No.: US 2010/0055730 A1
(43) Pub. Date: Mar. 4, 2010

(54) Methods for the Diagnosis and Treatment of Female Infertility Using Molecular Markers

(76) Inventors: Anny Usheva-Simidjiyska, Brookline, MA (US); Alan Penzias, Dedham, MA (US)

Correspondence Address: CLARK & ELBING LLP, 101 FEDERAL STREET, BOSTON, MA, 02110, US

(21) Appl. No.: 12/227,001

(22) PCT Filed: May 7, 2007
(86) PCT No.: PCT/US07/11014

§ 371 Date: Oct. 14, 2009

Related U.S. Application Data
(60) Provisional application No. 60/797,994, filed on May 5, 2006.
(60) Provisional application No. 60/918,338, filed on Mar. 16, 2007.

(51) Int. Cl. C12Q 1/02

(52) U.S. Cl. 435/29

(57) ABSTRACT

The present invention features methods of evaluating the fertility of a female subject, or the fertilization competence of an oocyte extracted from an ovarian follicle, using measurements of one or more differentially expressed components of follicular fluid. The methods of the invention include the steps of measuring the level of a component of follicular fluid. Examples of components of follicular fluid useful in the methods of the invention include apolipoprotein IA, apolipoprotein A, apolipoprotein B, apolipoprotein E, prothrombin, CD 133 (prominin), alpha-2 macroglobulin, alpha crystallin B chain, ATP synthase alpha chain, neuropilin, heparin, heparin-like molecules, heparin receptors, bile acids, Aid, CS, Cortisol, Ang1, Ang2, cholesterol and its derivatives, cholesterol receptors, phospholipids, HDL, LDL, VLDL5 chylomicrons, retinoids, carotenoids, retinol-binding proteins, retinoic acid receptors, transthyretins, leptin, fibrin, ADPases, metal ions, and cytokines, e.g., IL-1β, IL-4, IL-5, IL-6, IL-8, IL-IO, IL-12p40, IL-12p70, IL-13, VEGF, VEGF receptors, PlGF, INF-γ, Aid, CS, Ang1, Ang2, TNF-α, C-reactive protein, and angiopoetin, and comparing the measured level to a reference range in order to determine whether the subject is likely to be fertile, or whether the oocyte is likely to be fertilization-competent. The invention also features methods of treatment of an infertile subject, wherein the treatment methods utilize measurements of one or more differentially expressed components of follicular fluid to determine optimal conditions for performing assisted reproductive therapy, e.g., in vitro fertilization.

### BACKGROUND OF THE INVENTION

In general, this invention relates to the field of fertility.

A major reason women seek assisted reproductive technologies is for treatment of reduced fertility. Presently, a woman's age is the single most significant factor for the prediction of fertility success with ovulation induction and in vitro fertilization (IVF) because age is closely correlated with ovarian reserve. However, fertilization competence differs among similarly aged women as do the oocytes isolated from these women, and the mechanisms responsible for these differences are poorly understood. Thus, women undergoing assisted reproductive therapy often endure multiple failed attempts to become pregnant, resulting in high economic and emotional costs. There is a need to better understand the mechanisms responsible for fertility success or failure and to identify factors correlated with this success or failure in order to develop improved methods of evaluation, diagnosis, and therapy.

### SUMMARY OF THE INVENTION

Follicular fluid composition and follicular somatic cells play an important role in the maturation of the developing oocyte within the female ovary (FIG. 1). Although the molecular composition of follicular fluid is an indicator of the cellular and oocyte activities in the follicle, follicular fluid composition has been poorly characterized to date. The present invention provides follicle-specific markers of ovarian function that can be used for diagnosis and treatment of fertility disorders. These markers include, but are not limited to, apolipoprotein (e.g., 1A, A, B, or E), prothrombin, CD133 (prominin), macroglobulins (e.g., alpha-2 macroglobulin), alpha crystallin B chain, ATP synthase alpha chain, neuropilin, heparin, heparin-like molecules, heparin receptors, bile acids, aldosterone (Ald), corticosterone (CS), cortisol, angiotensin I (Ang1), angiotensin II (Ang2), cholesterol (e.g., esters or derivatives), cholesterol receptors, phospholipids, high-density lipoprotein (HDL), low-density lipoprotein (LDL), very low-density lipoprotein (VLDL), chylomicrons, retinoids, carotenoids, retinol-binding proteins, retinoic acid receptors, transthyretins, leptin, fibrin, ADPases, metal ions, or a cytokine, e.g., IL-1β, IL-4, IL-5, IL-6, IL-8, IL-10, IL-12p40, IL-12p70, IL-13, vascular endothelial growth factor (VEGF), VEGF receptors, placental growth factor (PlGF), interferon gamma (INF-γ), tumor necrosis factor alpha (TNF-α), C-reactive protein, and angiopoetin. For example, higher levels of apolipoprotein 1A or prothrombin are correlated with increased fertility and/or more favorable assisted reproductive therapy outcome, while lower levels of apolipoprotein 1A or prothrombin are correlated with decreased fertility and/or less favorable assisted reproductive therapy outcome. In addition, higher levels of IL-13 or VEGF are correlated with decreased fertility and/or less favorable assisted reproductive therapy outcome, while lower levels of IL-13 or VEGF are correlated with increased fertility and/or more favorable assisted reproductive therapy outcome.

Accordingly, in one aspect, the invention features a method of evaluating the fertilization competence of an oocyte extracted from an ovarian follicle by measuring the level (e.g., the concentration) of a component (e.g., apolipoprotein 1A or prothrombin) present in follicular fluid associated with the oocyte and comparing the level of the component to a reference range, in which a measurement of the level of the component within the reference range indicates that the oocyte is likely to be fertilization-competent. In an embodiment, the component is apolipoprotein 1A or prothrombin, and an increase in the level of apolipoprotein 1A or prothrombin present in the follicular fluid tested relative to the level of apolipoprotein 1A or prothrombin present in follicular fluid associated with a fertilization-incompetent oocyte indicates that the oocyte being evaluated is likely to be fertilization-competent. In other embodiments, the component is IL-13 or VEGF, and a decrease in the level of IL-13 or VEGF present in the follicular fluid tested relative to the level of IL-13 or VEGF present in follicular fluid associated with a fertilization-incompetent oocyte indicates that the oocyte being evaluated is likely to be fertilization-competent.

In another aspect, the invention features a method of evaluating the fertilization competence of an oocyte extracted from an ovarian follicle by measuring the level (e.g., the concentration) of a component present in follicular fluid and comparing the level of the component to a reference range, in which a measurement of the level of the component outside of the reference range indicates that the oocyte is likely to be fertilization-incompetent. In some embodiments, the component can include apolipoprotein 1A or prothrombin, and a decrease in the level of apolipoprotein 1A or prothrombin relative to a level of apolipoprotein 1A or prothrombin associated with a fertilization-competent oocyte indicates that the oocyte being evaluated is likely to be fertilization-incompetent. In other embodiments, the component can include IL-13 or VEGF, and an increase in the level of IL-13 or VEGF relative to a level of IL-13 or VEGF associated with a fertilization-competent oocyte indicates that the oocyte being evaluated is likely to be fertilization-incompetent.

In still another aspect, the invention features a method of evaluating the fertility of a female subject by measuring the level (e.g., the concentration) of a component of follicular fluid from the subject and comparing the level to a reference range, wherein a measurement of the level within the reference range indicates that the subject is likely to be fertile. In some embodiments, the component can include apolipoprotein 1A or prothrombin, and an increase in the level of apolipoprotein 1A or prothrombin relative to a level of apolipoprotein 1A or prothrombin associated with an infertile female subject indicates that the subject being evaluated is likely to be fertile. In other embodiments, the component can include IL-13 or VEGF, and a decrease in the level of IL-13 or VEGF relative to a level of IL-13 or VEGF associated with an infertile female subject indicates that the subject being evaluated is likely to be fertile.

In yet another aspect, the invention features a method of evaluating the fertility of a female subject by measuring the level (e.g., concentration) of a component of follicular fluid from the subject and comparing the level to a reference range, wherein a measurement of the level outside of the reference range indicates that the subject is likely to be infertile. In some embodiments, the component can include apolipoprotein 1A or prothrombin, and a decrease in the level of apolipoprotein 1A or prothrombin relative to a level of apolipoprotein 1A or prothrombin associated with a fertile female subject indicates that the subject being evaluated is likely to be infertile. In other embodiments, the component can include IL-13 or VEGF, and an increase in the level of IL-13 or VEGF relative to a level of IL-13 or VEGF associated with a fertile female subject indicates that the subject being evaluated is likely to be infertile.

In various embodiments of any of the above aspects, the subject can be human, or can be a non-human mammal, e.g., a cow, a horse, a sheep, a pig, a goat, a dog, or a cat.

In various embodiments of any of the above aspects, the measurement may include the steps of: (a) obtaining a sample that includes one or more components of follicular fluid; (b) running the sample in a two-dimensional gel to resolve the component(s) by pH and molecular weight; (c) staining the gel in order to visualize the component(s); and (d) determining the presence or absence of the component(s) in the gel. In some embodiments, the measurement includes quantifying the component(s) in the gel. Any technique for measuring the presence or concentration of the component(s) in a sample may be used, e.g., any antibody assay, e.g., ELISA; fluorescence polarization assays; mass spectrometry; surface plasmon resonance; fluorescence resonance energy transfer (FRET); bioluminescence resonance energy transfer (BRET); fluorescence quenching; fluorescence activated cell scanning/sorting; radioimmunoassay (RIA); or Coomassie staining and visual inspection of a 2D polyacrylamide gel.

In various embodiments of any of the above aspects, the reference range can include values greater than or equal to a reference value; alternatively, the reference range can include values less than or equal to a reference value.

In various embodiments of any of the above aspects, the component of follicular fluid to be assayed can be, e.g., apolipoprotein 1A, apolipoprotein A, apolipoprotein B, apolipoprotein E, prothrombin, CD133 (prominin), alpha-2 macroglobulin, alpha crystallin B chain, ATP synthase alpha chain, neuropilin, heparin, heparin-like molecules, heparin receptors, bile acids, Ald, CS, cortisol, Ang1, Ang2, cholesterol and its derivatives, cholesterol receptors, phospholipids, HDL, LDL, VLDL, chylomicrons, retinoids, carotenoids, retinol-binding proteins, retinoic acid receptors, transthyretins, leptin, fibrin, ADPases, or metal ions. In other embodiments, the component includes a cytokine, e.g., IL-1β, IL-4, IL-5, IL-6, IL-8, IL-10, IL-12p40, IL-12p70, IL-13, VEGF, VEGF receptors, PlGF, INF-γ, Ald, CS, Ang1, Ang2, TNF-α, C-reactive protein, or angiopoetin. In another embodiment, detection, in the follicular fluid accompanying an oocyte, of a level of one or more of the components indicated above that falls within the reference ranges listed in Table 1 indicates that the oocyte is likely fertilization competent.

In one embodiment, the component includes apolipoprotein 1A, the measurement includes determining the amount of the component that is present, and the reference range includes values greater than or equal to a reference value. The reference value can be, e.g., the minimum amount of the component that is detectable, e.g., by an antibody assay or Coomassie staining and visual inspection of a 2D gel.

In another embodiment, the component includes prothrombin, the measurement includes determining the amount of the component that is present, and the reference range includes values greater than or equal to a reference value. The reference value can be, e.g., the minimum amount of the component that is detectable, e.g., by an antibody assay or Coomassie staining and visual inspection of a 2D gel.

In still another embodiment, the component includes VEGF, the measurement includes determining the amount of the component that is present, and the reference range includes values less than or equal to a reference value.

In yet another embodiment, the component includes IL-13, the measurement includes determining the amount of the component that is present, and the reference range includes values less than or equal to a reference value.

In another aspect, the invention features a method of identifying a component of follicular fluid as a differentially expressed molecular marker, including the following steps: (a) obtaining a sample comprising the component of follicular fluid; (b) measuring the level of the component in the sample; and (c) comparing the measurement to a reference range, wherein a measurement of the level outside of the reference range indicates that the component is a differentially expressed molecular marker.

In another embodiment, the invention features a diagnostic test kit that may include components to perform any of the diagnostic methods described herein. The kit may include a binding agent, e.g., an antibody, that recognizes one or more components of follicular fluid (e.g., one or more of the components listed above).

In yet another embodiment, the invention features a device suitable for detecting on or more differentially expressed molecular markers of follicular fluid. In an embodiment, the molecular marker is selected from one or more of the components listed above.

By “component of follicular fluid” is meant any cells, cellular components, molecules, or molecular complexes from follicular fluid, e.g., proteins, peptides, lipids, lipoproteins, hormones, nucleic acids, or carbohydrates, that are present within an ovarian follicle. For example, follicular fluid can include secretions from granulosa cells and diffusion from theca cells. In particular, a follicular fluid component includes, e.g., apolipoprotein (e.g., 1A, A, B, or E), prothrombin, CD133 (prominin), macroglobulins (e.g., alpha-2 macroglobulin), alpha crystallin B chain, ATP synthase alpha chain, neuropilin, heparin, heparin-like molecules, heparin receptors, bile acids, aldosterone (Ald), corticosterone (CS), cortisol, angiotensin I (Ang1), angiotensin II (Ang2), cholesterol (e.g., esters or derivatives), cholesterol receptors, phospholipids, high-density lipoprotein (HDL), low-density lipoprotein (LDL), very low-density lipoprotein (VLDL), chylomicrons, retinoids, carotenoids, retinol-binding proteins, retinoic acid receptors, transthyretins, leptin, fibrin, ADPases, metal ions, or a cytokine, e.g., IL-1β, IL-4, IL-5, IL-6, IL-8, I-10, IL-12p40, IL-12p70, IL-13, vascular endothelial growth factor (VEGF), VEGF receptors, placental growth factor (PlGF), interferon gamma (INF-γ), tumor necrosis factor alpha (TNF-α), C-reactive protein, and angiopoetin.

By “differentially expressed molecular marker” is meant a cellular component, molecule, or molecular complex that is present at different levels, e.g., substantially different levels, in different subjects or samples. For example, a molecule that is present at a higher or lower concentration in follicular fluid from a fertilization-competent oocyte relative to follicular fluid from a fertilization-incompetent oocyte is a differentially expressed molecular marker. Such a molecule can be used in a method for diagnosing and/or treating a condition, e.g., infertility, by detecting the molecule using one or more of the methods described herein. Alternatively, the molecule can be used to collect information about the status of a patient (e.g., the fertilization-competence of an oocyte of the patient).

By “fertile” is meant having the ability to conceive and have offspring, e.g., after a year of regular sexual intercourse without contraception.

By “fertilization-competent” in the context of an oocyte is meant an oocyte that is capable of becoming fertilized, e.g., through sexual intercourse or through any assisted reproduction therapy, e.g., in vitro fertilization.

By “fertilization-incompetent” in the context of an oocyte is meant an oocyte having a reduced capability or an oocyte that is incapable of becoming fertilized, e.g., through sexual intercourse or through any assisted reproduction therapy, e.g., in vitro fertilization.

By “infertile” is meant having a diminished ability, or inability, to conceive and have offspring, e.g., after a year of regular sexual intercourse without contraception.

By “likely” in the context of diagnosis or evaluation is meant having a substantial probability of being true, e.g., at least 30%, 40%, 50%, 60%, 70%, 80%, 90%, 95%, or even 99% likely.

By “measurement” is meant any experiment or assay, e.g., a clinical assay, that generates one or more values or numbers.

By “reference range” is meant any range of values, including a single value, that is used for purposes of comparison. A reference range can reflect the outcome of a single control experiment or assay, or it can be a statistical function of multiple experiments or assays. A reference range can also reflect the presence or absence of a signal, or the relative frequency of a signal in a given population. For example, a reference range can include a percentage, or range of percentages, of samples in which a given peptide spot is present in a 2D gel. A reference range can be generated, e.g., from a prior measurement from the same subject or a measurement from a fertile or infertile subject or from a fertilization-competent or fertilization-incompetent oocyte. A reference range can also be generated by measuring a purified reference substance, e.g., a protein, at a known concentration and establishing a value within ±0.1, 1, 2, 5, 10, 15, 20, 30, 50, 75, 100, 200, 250, 500, 1000, or 10,000%, or more of the detected concentration.

By “reference value” is meant a single value within a reference range.

By “subject” is meant a mammal, including, but not limited to, a human or non-human mammal, such as a cow, a horse, a sheep, a pig, a goat, a dog, or a cat.

The methods of the invention offer multiple improvements and advantages over previously available methods of diagnosis or treatment. For example, the invention provides previously unknown folliculogenesis and oogenesis marker molecules that are useful for prediction of oocyte viability, oocyte fertility competence, and oocyte embryogenesis competence; evaluation of human fertility and infertility; identification of unexplained infertility; and measurement of age-related fertility decline. The marker molecules identified herein are also useful as targets for therapeutic drug discovery (e.g., contraceptives or fertility-inducing compounds), reversible blocking of follicular maturation, treatment of unexplained and age-related infertility, and modification of implantation rate, as well as for toxicity screens.

Other features and advantages of the invention will be apparent from the detailed description and from the claims.

### BRIEF DESCRIPTION OF THE DRAWINGS

FIG. 1 is a schematic illustration of a preovulatory follicle.

FIG. 2 is a flowchart of a protocol for processing follicular fluid samples. In this protocol, samples are subjected to protein A/G chromatography to remove immunoglobulins. The remaining fluid is TCA precipitated and centrifuged, and pelleted proteins are subjected to sonication and 2D gel electrophoresis in duplicate. Differentially expressed protein spots are analyzed further.

FIG. 3 is a set of images of 2D gels generated by the protocol outlined in FIG. 2. Gels corresponding to young lead (upper left), young small (upper right), old lead (lower left), and old small (lower right) follicles are shown. In each case, the horizontal axis represents pH, while the vertical axis represents molecular weight in kilodaltons. Several differentially expressed protein spots are indicated: 1 corresponds to prothrombin, 2 corresponds to alpha crystallin B chain (molecular weight 20.1 kDa, lp 7.4), and 3 corresponds to ATP synthase alpha chain (MW 50.9 kDa, lp 6.8).

FIG. 4 is an image of a 2D gel corresponding to a reproductively young subject. The peptide spots in the area labeled “A” are present in a greater percentage of young subjects in comparison to old subjects. The peptide spots in the area labeled “B” are likewise differentially expressed in favor of young subjects.

FIG. 5 is an image of a 2D gel corresponding to a reproductively old subject. The peptide spots in the area labeled “A” are present in a greater percentage of old subjects in comparison to young subjects.

FIG. 6 is a chart showing that the pregnancy rate for young subjects is higher than the pregnancy rate for old subjects.

FIG. 7 is an image of a representative 2D gel. The peptide spot labeled “a,” which corresponds to apolipoprotein 1A, is present in a greater percentage of young subjects in comparison to old subjects, and in a greater percentage of pregnant subjects in comparison to non-pregnant subjects.

FIG. 8A is a graph comparing the mean concentration of aldosterone, angiotensin I, and angiotensin II in large (Lf) and small (Sf) follicles. FIG. 8B is a graph comparing the mean concentration of corticosterone in large and small follicles.

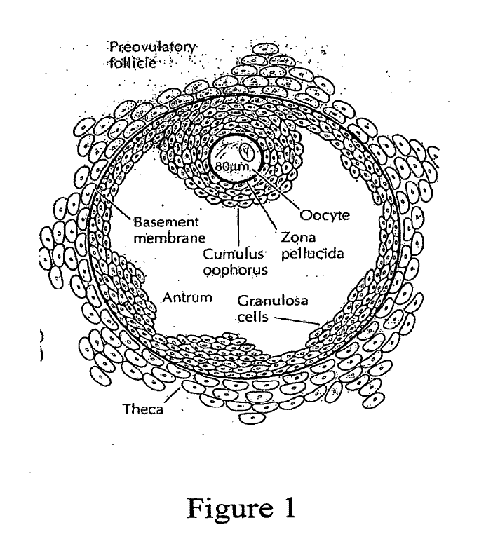

### DETAILED DESCRIPTION OF THE INVENTION

We have discovered that certain molecular markers found in follicular fluid are differentially expressed in distinct subject populations, e.g., fertile versus infertile, pregnant versus non-pregnant, or reproductively young versus reproductively old. By comparing measurements of one or more components of follicular fluid between two subjects or groups, e.g., a test subject and a control subject, we can assess the likelihood of fertility in a subject or fertilization competence in an oocyte. These assessments, in turn, may be used to guide assisted reproductive therapy, e.g., to increase the probability of successful fertilization, implantation, and fetal development.

While the detailed description refers herein to specific molecular markers, e.g., apolipoprotein 1A, apolipoprotein A, apolipoprotein B, apolipoprotein E, prothrombin, CD133 (prominin), alpha-2 macroglobulin, alpha crystallin B chain, ATP synthase alpha chain, neuropilin, heparin, heparin-like molecules, heparin receptors, bile acids, Ald, CS, cortisol, Ang1, Ang2, cholesterol and its derivatives, cholesterol receptors, phospholipids, HDL, LDL, VLDL, chyloricrons, retinoids, carotenoids, retinol-binding proteins, retinoic acid receptors, transthyretins, leptin, fibrin, ADPases, metal ions, and cytokines, e.g., IL-1β, IL-4, IL-5, IL-6, IL-8, IL-10, IL-12p40, IL-12p70, IL-13, VEGF, VEGF receptors, PlGF, INF-γ, Ald, CS, Ang1, Ang2, TNF-α, C-reactive protein, and angiopoetin, it will be clear to one of skill in the art that the detailed description can also apply to family members, isoforms, and/or variants of these or other molecular markers found in follicular fluid.

### Diagnostics

In general, the methods of diagnosis described herein feature measuring the level of a component of follicular fluid isolated from the ovarian follicle of a subject and comparing the measured level to a reference range. In some instances, a measurement of the level within the reference range indicates an increased likelihood of subject fertility or oocyte fertilization competence, whereas a measurement of at least 0.01, 0.1, 1, 5, 10, 15, 20%, or more outside of the reference range indicates a decreased likelihood of subject fertility or oocyte fertilization competence.

Based on the results described herein, in which numerous components of follicular fluid are shown to be differentially expressed, i.e., expressed at different levels in different subject or oocyte populations, one or more suitable reference ranges can be determined for each marker and used as a basis for diagnostic comparison. For example, we have found that apolipoprotein 1A is more abundant in reproductively young (e.g., age 34 or younger) or pregnant subjects in comparison to reproductively old (e.g., age 40 or over) or non-pregnant subjects, respectively. A suitable reference range for apolipoprotein 1A can include, e.g., any measured value greater than or equal to a reference value, or any value between two reference values. In one instance, the measurement detects the level of apolipoprotein 1A in a sample, e.g., by using an antibody-based assay such as ELISA, or by using Coomassie staining and visual inspection of a 2D polyacrylamide gel; the reference range could include, e.g., any level greater than or equal to the minimal detectable level using the chosen method of measurement, or any level greater than or equal to another reference level. Measurements falling outside the reference range would indicate a likelihood of infertility, whereas measurements falling within the reference range would indicate a likelihood of fertility. Any suitable method of measurement may be used, examples of which are described herein.

Any differentially expressed marker found within follicular fluid may be used as the basis of a diagnostic assay. For example, the methods of the invention may utilize, but are not restricted to utilizing, the following molecular markers: apolipoprotein LA, apolipoprotein A, apolipoprotein B, apolipoprotein E, prothrombin, CD133 (prominin), alpha-2 macroglobulin, alpha crystallin B chain, ATP synthase alpha chain, neuropilin, heparin, heparin-like molecules, heparin receptors, bile acids, Ald, CS, cortisol, Ang1, Ang2, cholesterol and its derivatives, cholesterol receptors, phospholipids, HDL, LDL, VLDL, chylomicrons, retinoids, carotenoids, retinol-binding proteins, retinoic acid receptors, transthyretins, leptin, fibrin, ADPases, metal ions, or cytokines, e.g., IL-1β, IL-4, IL-5, IL-6, IL-8, IL-10, IL-12p40, IL-12p70, IL-13, VEGF, VEGF receptors, PlGF, INF-γ, Aid, CS, Ang1, Ang2, TNF-α, C-reactive protein, or angiopoetin. In general, any component of follicular fluid that is differentially present based on follicular size, fertility competence, rate of implantation, and/or age, can be utilized in the invention. In addition, any difference in post-translational protein modification, e.g., phosphorylation, glycosylation, methylation, acetylation, lipid modification, ubiquitination, hydroxylation, carboxylation, or post-translational proteolysis, can be utilized in the invention.

Antibodies to the differential markers described herein are known in the art. In addition, methods of detection of these markers are known in the art, e.g., involving the use of antibodies to the markers.

Any suitable reference range may be used in conjunction with measurements of differentially expressed molecular markers; for example, a reference range could include any value less than or equal to a reference value, e.g., a threshold of detectability; any value greater than or equal to a reference value; and any value between two predetermined reference values. The choice of reference range can be determined empirically, theoretically, or a combination of both.

Exemplary reference ranges are listed in Table 1 and are not intended to be limiting in any way. Each of the lower and upper bounds in any given exemplary reference range can vary by, e.g., a factor of 1.1, 1.2, 1.3, 1.4, 1.5, 2, 3, 4, 5, 10, 20, 30, 40, 50, 100, 200, 500, 1000, 2000, 5000, 10⁴, 10⁶, or 10⁷ less than or greater than the lower or upper bounds of the reference ranges listed in Table 1.

TABLE 1

| | Reference ranges of exemplary | |
|---|---|---|
| Molecular Marker | molecular markers in follicular fluid | |
| VEGF and VEGF isoforms | 0.001-500 | ng/ml |
| VEGF receptors | 0.001-500 | ng/ml |
| Neuropilin | 0.001-500 | ng/ml |
| PlGF | 0.001-500 | ng/ml |
| C-reactive protein | 0-500 | mg/L |
| Angiopoetin | 0.1-100,000 | pg/ml |
| Heparin and heparin-like molecules | 0.001-1,000 | IU/ml |
| Hyaluronans | 0.001-2,000 | IU/ml |
| Ang1 and Ang2 | 0.001-1,000 | U/ml |
| Ald | 0.01-10,000 | pg/ml |
| Cortisol | 0.001-9,000 | ng/ml |
| CS | 0.001-2,000 | ng/ml |
| Apolipoprotein A | 0.001-500 | mg/ml |
| Apolipoprotein B | 0.001-500 | mg/ml |
| Apolipoprotein E | 0.001-500 | mg/ml |
| Bile acids | 0.001-1,000 | μM |
| Cholesterol, cholesterol ester, and | 0.001-500 | mg/ml |
| cholesterol derivatives | | |
| Phospholipids | 0.001-500 | mg/cc |
| HDL | 0.01-500 | mg/ml |
| LDL | 0.01-500 | mg/ml |
| VLDL | 0.01-500 | mg/ml |
| Chylomicrons | 0.01-500 | mg/ml |
| Cytokines | 0.001-9,000 | pmol |
| Retinoids and carotenoids | 0.001-10,000 | μg/dl |
| Retinol-binding protein | 0.001-100 | mg/ml |
| Transthyretin | 0.001-100 | mg/ml |
| Retinoic acid receptors | 0.01-9,000 | fmol |
| Leptin | 0.001-1,000 | pg/l |
| Macroglobulins | 0.1-500 | rU |
| Prominin | 0.01-10,000 | fmol |
| Metal ions | 0.001-10,000 | pmol |
| ADPases | 0.001-10,000 | U/ml |

Any suitable method of measurement may be used, e.g., antibody assays, ELISA, fluorescence polarization assays, mass spectrometry (Nelson and Krone, J. Mol. Recognit., 12:77-93, 1999), surface plasmon resonance (Spiga et al., FEBS Lett., 511:33-35, 2002; Rich and Mizka, J. Mol. Recognit., 14:223-228, 2001; Abrantes et al., Anal. Chem., 73:2828-2835, 2001), fluorescence resonance energy transfer (FRET) (Bader et al., J. Biomol. Screen, 6:255-264, 2001; Song et al., Anal. Biochem. 291:133-41, 2001; Brockhoff et al., Cytometry, 44:338-248, 2001), bioluminescence resonance energy transfer (BRET) (Angers et al., Proc. Natl. Acad. Sci. USA, 97:3684-3689, 2000; Xu et al., Proc. Natl. Acad. Sci. USA, 96:151-156, 1999), fluorescence quenching (Engelborghs, Spectrochim. Acta A. Mol. Biomol. Spectrosc., 57:2255-2270, 1999; Geoghegan et al., Bioconjug. Chem. 11:71-77, 2000), fluorescence activated cell scanning/sorting (Barth et al., J. Mol. Biol., 301:751-757, 2000), radioimmunoassay (RIA), or Coomassie staining and visual inspection of a 2D polyacrylamide gel.

The measurement of any of the components of follicular fluid described herein can occur on one or more occasions, and a change in the measured levels over time can be used as an indicator of development of increased or decreased subject fertility or oocyte fertilization competence.

The diagnostic methods described herein can be used individually or in combination with any other diagnostic method described herein or determined to be useful for a more accurate diagnosis of fertility or infertility.

The diagnostic methods described herein can also be used to monitor and manage a subject's fertility or an oocyte's fertilization competence. In one instance, the levels of one or more components of a subject's follicular fluid are monitored until one or more favorable measurements are made, e.g., one or more measurements are found to be within a suitable reference range. Such a method allows for prediction or determination of appropriate times to attempt fertilization of a subject or oocyte.

### Diagnostic Kits and Devices

The invention also provides for diagnostic test kits and devices. For example, a diagnostic test kit can include a binding agent, e.g., an antibody, to one or more components of follicular fluid. In one example, the kit includes an antibody that specifically binds apolipoprotein 1A. A conventional ELISA is a common, art-known method for detecting antibody-substrate interaction and can be provided with the kit of the invention.

Desirably, a kit includes any of the components needed to perform any of the diagnostic methods described above. For example, the kit desirably includes a membrane, where the agent that binds the component of follicular fluid, or a secondary binding agent (e.g., an agent that binds a follicular fluid component-binding agent) is immobilized on the membrane. The membrane can be supported on a dipstick structure where the sample is deposited on the membrane by placing the dipstick structure into the sample, or the membrane can be supported in a lateral flow cassette in which the sample is deposited on the membrane through an opening in the cassette.

A diagnostic kit of the invention also generally includes a label or instructions for the intended use of the kit components, as well as a reference sample or purified proteins to be used to establish a standard curve. In one example, the kit contains instructions for the use of the kit for the diagnosis of fertility or infertility. In yet another example, the kit contains instructions for the use of the kit to monitor therapeutic treatment or dosage regimens for the treatment of infertility. It will be understood that the reference sample values will depend on the intended use of the kit. For example, the measured value or values can be compared to a normal reference value or a reference range, as described herein.

The invention also provides for a device suitable for detecting differentially expressed molecular markers present in follicular fluid. The device may contain, e.g., a membrane having antibodies bound thereto which is suitable for contact with a liquid, e.g., follicular fluid. Exemplary devices are listed in, e.g., U.S. Pat. Nos. 4,033,723, 5,786,220, and 6,558,897, hereby incorporated by reference.

### Therapy

Any of the methods described herein can be utilized to collect information about a patient or for diagnostic purposes in order to guide assisted reproductive therapy, e.g., ovulation induction, in vitro fertilization with subject oocytes, in vitro fertilization with donor oocytes, cryoembryo transfer, or intracytoplasmic sperm injection. Any follicular fluid differential markers (such as the components described herein) can be used to determine optimal conditions for carrying out one or more assisted reproductive therapies, or can be used to select a suitable method of therapy. For example, using a diagnostic method of the invention, one can assay a component of follicular fluid from multiple follicles, using the results of the diagnostic assay to choose one or more oocytes that are likely to be fertilization-competent. Such an approach can substantially reduce the failure rate of subsequent assisted reproductive therapy, e.g., in vitro fertilization. In addition, follicular fluid can be isolated from a subject at multiple time points and assayed; the results of these assays can be used to determine the best time or times to carry out assisted reproductive therapy.

Therapy according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the degree of the subject's infertility, and how the patient responds to the treatment.

### EXAMPLES

The following examples are provided for the purpose of illustrating the invention and are not meant to limit the invention in any way.

### Example 1

### Human Follicular Fluid Composition Correlates with Age and Fertility Potential

The experiments described in this example were designed to determine whether follicular fluid composition is correlated with ovarian aging, follicle maturity, fertility potential, and/or in vitro fertilization outcome.

We recruited infertility patients undergoing in vitro fertilization on the day of their egg retrieval. A total of 37 women were included, and they were classified as reproductively young (age 34 or younger) or reproductively old (age 40 or older). Patients were included if they had a diagnosis of unexplained infertility, male factor infertility, or an ovulatory disorder. Only patients undergoing their first or second in vitro fertilization cycle were included; thus, poor responders were excluded.

The stimulation protocol was standard and included down-regulation with either a GnRH agonist or antagonist, hyperstimulation with recombinant FSH, hCG as an LH surrogate to trigger ovulation, and vaginal or IM progesterone for luteal support.

On the day of egg retrieval, an ultrasound was used to measure all follicles prior to aspiration. A follicle was labeled “lead,” i.e., mature, if it had a diameter of 14 mm or greater. It was labeled “small,” i.e., immature, if the diameter was 12 mm or less. Follicular fluid was then collected in vials. Each vial contained the pooled follicular fluid from either three lead or three small follicles. The oocytes were removed by an embryologist, and the follicular fluid was centrifuged to separate cellular components.

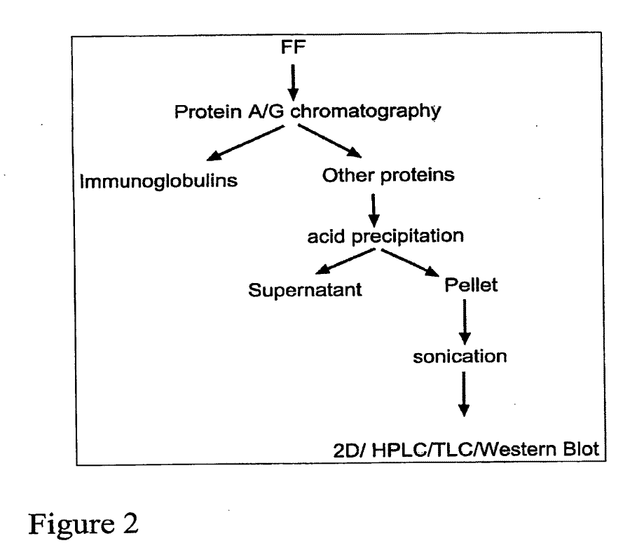

A protocol was then developed for preparing the follicular fluid samples for several experiments (FIG. 2). The samples were first subjected to protein A/G chromatography to remove some of the immunoglobulins. The remaining fluid was prepared with a TCA precipitation. The condensed proteins were then subjected to sonication, and 2D gel electrophoresis was performed in duplicate on all samples. Finally, the differentially expressed protein spots were visually identified using Coomassie stain, excised, and subjected to mass spectrometry to confirm the identity of the peptides.

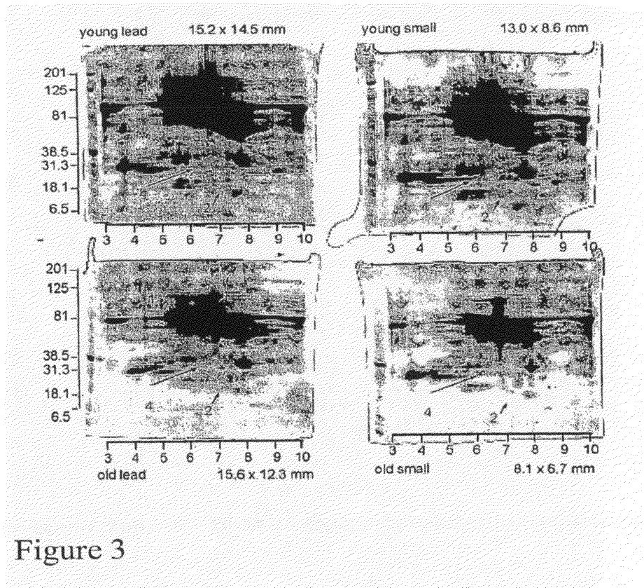

Gels for several classes of samples were run, including young lead (FIG. 3, upper left), young small (FIG. 3, upper right), old lead (FIG. 3, lower left), and old small (FIG. 3, lower right) follicle samples. By using the molecular weight and pH grids, it was possible to visually match differentially expressed protein spots to control gels. For example, spot #1 is most intense in young lead follicles and is identical to the prothrombin protein spot seen in control 2D maps.

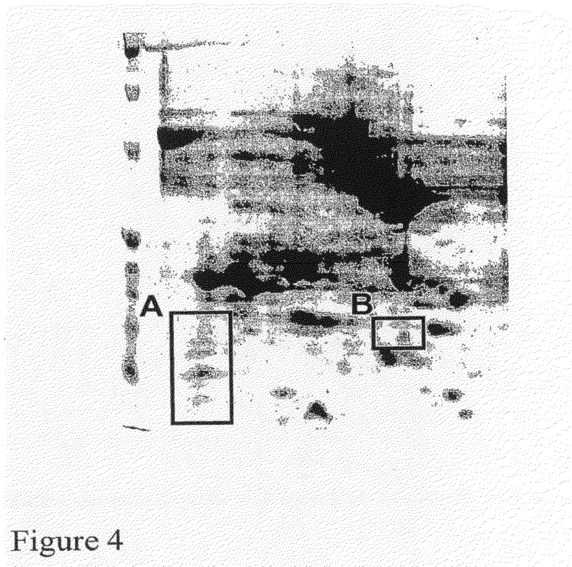

We next examined the general profile of a reproductively young patient. Several proteins were observed to be differentially expressed between young and old patients. Several protein spots having a pH of approximately 4.0 and molecular weight of 6-14 kDa (FIG. 4, region “A”) were present in 65% of the younger patients and in only 41% of the older patients. A protein spot having a pH of 7.8 and molecular weight of 18 kDA (FIG. 4, region “B”) was present in 60% of young patients and in only 24% of old patients.

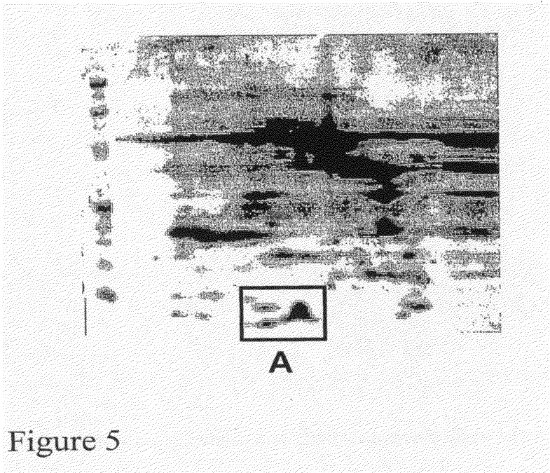

We also examined the general profile of a reproductively old patient. Several protein spots having a pH of approximately 6.0 and a molecular weight of 5 kDA (FIG. 5, region “A”) were present in 65% of old patients and 25% of young patients.

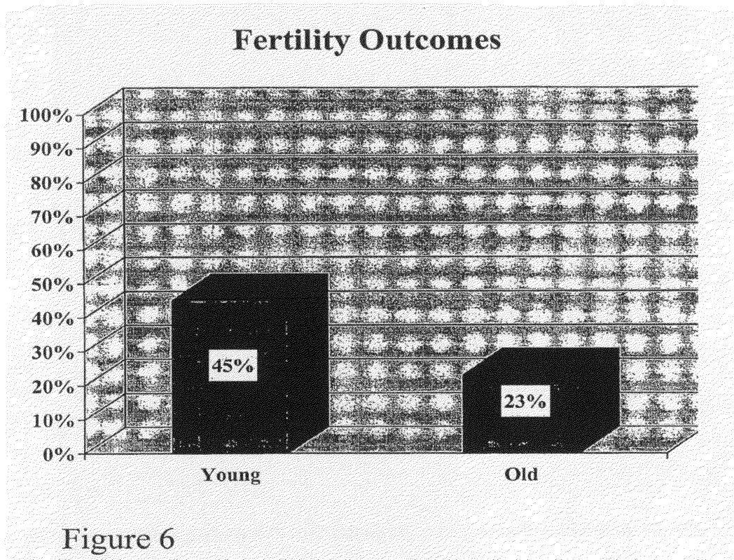

When fertility outcomes were evaluated in general, there was a clear association between pregnancy rates and age. Specifically, the pregnancy rate for young patients was 45%, compared to 23% in old patients (FIG. 6).

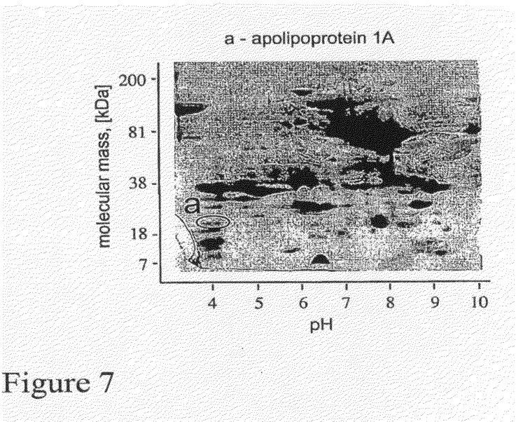

In addition, we identified a differentially expressed protein with a pH of 4.2 and molecular weight of 23 kDa. This protein was present in 65% of the younger patients compared to 35% of the older patients. In addition, it was present in 77% of the pregnant patients compared to only 17% of the non-pregnant patients (chi square, p<0.001). When this protein spot was subjected to mass spectrometry for identification, it was identified as apolipoprotein 1A, a high-density lipoprotein (FIG. 7, region “a”).

In conclusion, the results described herein indicate that the specific microenvironment of the developing follicle can influence oocyte maturation and fertility potential. In particular, we observed aging-specific variation in follicular fluid composition; in addition, differences in follicular fluid composition and aging were associated with variation in pregnancy rates. Furthermore, a specific follicular fluid component, apolipoprotein 1A, was found to be highly correlated with lead follicles of young patients who ultimately became pregnant. Additional characterization and identification of follicular fluid components by mass spectrometry is likely to provide additional novel biomedical markers of oocyte competence and fertility potential.

### Example 2

### Cytokines in Follicular Fluid and their Correlation with In Vitro Fertilization Outcome Measures

The experiments described in this example aimed to delineate the presence of cytokines within human ovarian follicular fluid and correlate their concentration with in vitro fertilization (IVF) outcome measures.

Patients undergoing IVF were recruited and consented at the time of oocyte retrieval according to an approved protocol. Follicular fluid was obtained after follicular measurement in two dimensions via transvaginal ultrasound with a mean diameter greater than 14 mm. Follicular fluid was centrifuged, and immunoglobulins were removed with A/G chromatography. Sixteen follicular fluid samples were analyzed for fourteen human cytokines using microarray technology. The analyses were repeated with two identical arrays. As a second assay, multiplex sandwich enzyme linked immunosorbent assay (ELISA) utilizing a robotic array of sixteen cytokines was performed. Duplicate analyses were conducted on twenty follicular fluid samples from separate patients. Data collected for each sample included patient age, baseline follicle-stimulating hormone, body mass index, infertility diagnosis, gravity, parity, cycle protocol, number of oocytes retrieved, number of mature oocytes, fertilization rates, number of embryos transferred and cryopreserved, pregnancy rates, and pregnancy outcome. Data were analyzed using correlation coefficients, chi-square, and Student's t-tests where appropriate, with a statistical significance set at p<0.05.

Multiple cytokines were found in follicular fluid in high concentrations, including Interleukins (IL)-1β, IL-4, IL-5, IL-6, IL-8, IL-10, IL-12p40, IL-12p70, and IL-13, and growth factors vascular endothelial growth factor (VEGF), placental growth factor (PlGF), interferon gamma (INF-γ), and tumor necrosis factor alpha (TNF-α). Cytokines were highly correlated (p<0.001) with each other, especially IL-4, IL-5, IL-6, IL-10, IL-12, IL-13, INF-γ, and TNF-α. VEGF and IL-8 were also highly correlated (p=0.028) with each other. IL-13 and VEGF were found to be significantly correlated with IVF outcome. As the IL-13 concentration increased, the number of oocytes retrieved, the number of mature oocytes, and the pregnancy rate was found to decline (p<0.05). Increased VEGF content in the follicular fluid was correlated with a decreased number of oocytes retrieved and a decreased number of mature oocytes (p<0.05).

In conclusion, the results described herein indicate that the cytokines within human ovarian follicular fluid play an important role in the follicular microenvironment. Furthermore, increased content of certain cytokines, such as IL-13 and VEGF, correlate with decreased in vitro fertilization success and may therefore be detrimental to the follicular/oocyte environment. These factors can be used as markers for IVF outcome.

### Example 3

### Aldosterones and Mineralcorticoids in Follicular Fluid and Their Correlation with the Maturation of Fertility-Competent Oocytes

The experiments described in this example aimed to determine the role of aldosterones and mineralcorticoids in the development of fertility-competent oocytes.

Follicular fluid from large (Lf) and small (Sf) follicles was obtained from women classified as reproductively young (age 34 or younger) or reproductively old (age 40 or older) who were undergoing in vitro fertilization (IVF). The follicular fluid samples were analyzed for protein content using an enzyme-linked immunosorbent assay (ELISA). Quantitative analysis using ELISA specifically screened for four aldosterones and mineralcorticoids, including Ald, CS, Ang1, and Ang2.

Elevated concentrations of aldosterone, corticosterone, and angiotensin II were found in the follicular fluid of large follicles (Table 2).

TABLE 2

| | Ald | CS | Ang2 |
|---|---|---|---|
| Concentration in Lf (pg/ml) | 241 (184) | 169,930 (83,825) | 468 (89) |
| (standard deviation) | | | |
| Plasma concentration | 10-30 | 900-3900 | 25-315 |
| (pg/ml) reference range | | | |

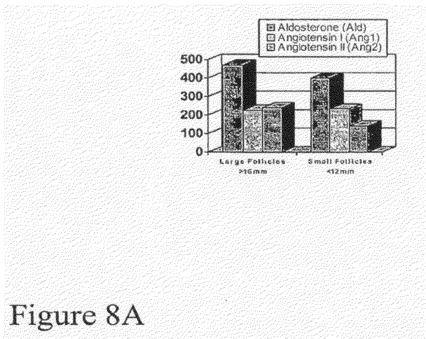

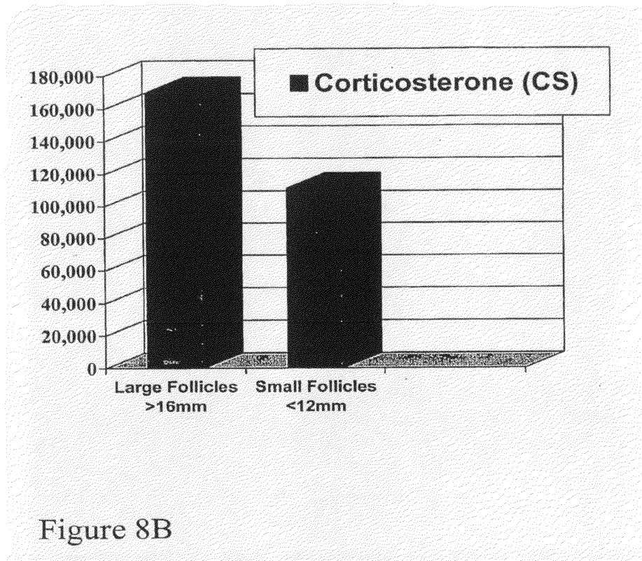

Follicular fluid from large follicles had a significantly increased mean concentration of corticosterone (p=0.02) and aldosterone (p=0.02) compared to the concentration in small follicles. A trend for an increased concentration of angiotensin II in large follicles was observed (p=0.08); however, no difference was observed in the concentration of angiotensin I between large and small follicles (FIGS. 8A and 8B). The concentration of corticosterone was positively correlated with the number of oocytes retrieved (R₂=0.413; p=0.45) and with the concentration of aldosterone in the follicular fluid (R₂=0.521; p=0.016). The concentration of angiotensin I was negatively correlated with age (R₂=0.559; p=0.38).

In conclusion, the results described herein demonstrate the presence of aldosterones and mineralcorticoids at high concentrations within the human ovary. Correlations with the number of oocytes retrieved suggest the involvement of aldosterones and mineralcorticoids in the maturation of fertility-competent oocytes.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of evaluating the fertilization competence of an oocyte extracted from an ovarian follicle, said method comprising measuring the level of a component of follicular fluid from said follicle and comparing said level to a reference range for said component, wherein a determination that the level of said component present in said follicular fluid is within the reference range indicates that said oocyte is likely to be fertilization-competent.

2. The method of claim 1, wherein said component comprises apolipoprotein 1A or prothrombin, and wherein an increase in said level of apolipoprotein 1A or prothrombin relative to a level of apolipoprotein 1A or prothrombin present in follicular fluid associated with a fertilization-incompetent oocyte indicates that said oocyte being evaluated is likely to be fertilization-competent.

3. The method of claim 1, wherein said component comprises IL-13 or vascular endothelial growth factor (VEGF), and wherein a decrease in said level of IL-13 or VEGF relative to a level of IL-13 or VEGF present in follicular fluid associated with a fertilization-incompetent oocyte indicates that said oocyte being evaluated is likely to be fertilization-competent.

4. A method of evaluating the fertilization competence of an oocyte extracted from an ovarian follicle, said method comprising measuring the level of a component of follicular fluid from said follicle and comparing said level to a reference range for said component, wherein a determination that the level of said component present in said follicular fluid is outside the reference range indicates that said oocyte is likely to be fertilization-incompetent.

5. The method of claim 4, wherein said component comprises apolipoprotein 1A or prothrombin, and wherein a decrease in said level of apolipoprotein 1A or prothrombin relative to a level of apolipoprotein 1A or prothrombin present in follicular fluid associated with a fertilization-competent oocyte indicates that said oocyte being evaluated is likely to be fertilization-incompetent.

6. The method of claim 4, wherein said component comprises IL-13 or VEGF, and wherein an increase in said level of IL-13 or VEGF relative to a level of IL-13 or VEGF present in follicular fluid associated with a fertilization-competent oocyte indicates that said oocyte being evaluated is likely to be fertilization-incompetent.

7. A method of evaluating the fertility of a female subject, said method comprising measuring the level of a component of follicular fluid from said subject and comparing said level to a reference range for said component, wherein a determination that the level of said component present in said follicular fluid is within the reference range indicates that said subject is likely to be fertile.

8. The method of claim 7, wherein said component comprises apolipoprotein 1A or prothrombin, and wherein an increase in said level of apolipoprotein 1A or prothrombin relative to a level of apolipoprotein 1A or prothrombin present in follicular fluid associated with an infertile female subject indicates that said subject being evaluated is likely to be fertile.

9. The method of claim 7, wherein said component comprises IL-13 or VEGF, and wherein a decrease in said level of IL-13 or VEGF relative to a level of IL-13 or VEGF present in follicular fluid associated with an infertile female subject indicates that said subject being evaluated is likely to be fertile.

10. A method of evaluating the fertility of a female subject, said method comprising measuring the level of a component of follicular fluid from said subject and comparing said level to a reference range, wherein a determination that the level of said component present in said follicular fluid is outside the reference range indicates that said subject is likely to be infertile.

11. The method of claim 10, wherein said component comprises apolipoprotein 1A or prothrombin, and wherein a decrease in said level of apolipoprotein 1A or prothrombin relative to a level of apolipoprotein 1A or prothrombin present in follicular fluid associated with a fertile female subject indicates that said subject being evaluated is likely to be infertile.

12. The method of claim 10, wherein said component comprises IL-13 or VEGF, and wherein an increase in said level of IL-13 or VEGF relative to a level of IL-13 or VEGF present in follicular fluid associated with a fertile female subject indicates that said subject being evaluated is likely to be infertile.

13. (canceled)

14. The method of claim 1, wherein said measurement comprises the steps of:
(a) obtaining a sample comprising one or more components of follicular fluid;
(b) running said sample in a two-dimensional gel to resolve said one or more components by pH and molecular weight;
(c) staining said gel in order to visualize said one or more components; and
(d) determining the presence or absence of said one or more components in said gel.

15. The method of claim 14, wherein said measurement further comprises quantifying said one or more components in said gel.

16. The method of claim 1, wherein said reference range comprises values greater than or equal to a reference value.

17. The method of claim 1, wherein said reference range comprises values less than or equal to a reference value.

18. The method of claim 1, wherein said component is selected from the group consisting of apolipoprotein 1A, apolipoprotein A, apolipoprotein B, apolipoprotein E, prothrombin, CD133 (prominin), alpha-2 macroglobulin, alpha crystallin B chain, ATP synthase alpha chain, neuropilin, heparin, heparin-like molecules, heparin receptors, bile acids, aldosterone (Ald), corticosterone (CS), angiotensin I (Ang1), angiotensin II (Ang2), cholesterol and its derivatives, cholesterol receptors, phospholipids, HDL, LDL, VLDL, chylomicrons, retinoids, carotenoids, retinol-binding proteins, retinoic acid receptors, transthyretins, leptin, fibrin, ADPases, and metal ions.

19. The method of claim 1, wherein said component comprises a cytokine.

20. The method of claim 19, wherein said cytokine is selected from the group consisting of IL-1β, IL-4, IL-5, IL-6, IL-8, IL-10, IL-12p40, IL-12p70, IL-13, VEGF, VEGF receptors, placental growth factor (PlGF), interferon gamma (INF-γ), tumor necrosis factor alpha (TNF-α), C-reactive protein, and angiopoetin.

21. The method of claim 18, wherein said component comprises apolipoprotein 1A or prothrombin, said measurement comprises determining the amount of said component that is present, and said reference range comprises values greater than or equal to a reference value.

22. The method of claim 21, wherein said reference value is the minimum amount of said component detectable by Coomassie staining and visual inspection of a 2D gel.

23. The method of claim 20, wherein said component comprises VEGF or IL-13, said measurement comprises determining the amount of said component that is present, and said reference range comprises values less than or equal to a reference value.

24-25. (canceled)

26. The method of claim 7, wherein said subject is human.

27. The method of claim 10, wherein said subject is human.
